(19) United States
(12) Patent Application Publication
(10) Pub. No.: US 2005/0152997 A1
(43) Pub. Date: Jul. 14, 2005

(54) All natural flavor enhancers for green tea beverages and dental hygiene product

(76) Inventors: Jonathan A. Selzer, Cheshire, CT (US); Cornelia D. Vasile, Middletown, CT (US)

Correspondence Address: John L. Cordani, Esq.;Carmody & Torrance LLP, P.O. Box 1110, 50 Leavenworth Street, Waterbury, CT, 06721-1110, US

(21) Appl. No.: 10/757,876

(22) Filed: Jan. 14, 2004

(51) Int. Cl. A61K 35/78

(52) U.S. Cl. 424/729; 424/757; 424/765

(57) ABSTRACT

A green tea based dietary supplement, nutritional beverage or dental hygiene product is revealed with enhanced flavor characteristics. Green tea sources are combined with extracts of the fruit of Lo Han and/or extracts from the leaves of Stevia rebaudiana and/or extracts from the leaves of Chinese Blackberry. The resulting product is a pleasant tasting dietary supplement, which is easily absorbed by the body in liquid form and provides substantially therapeutic effects.

### FIELD OF THE INVENTION

The present invention relates to flavored green tea beverages with improved taste and to a method for using such compositions as dietary supplements, therapeutic supplements and/or nutritional food beverages.

### BACKGROUND OF THE INVENTION

The use of tea products have many health benefits which are well documented in the scientific literature. Current research around the world is substantiating further claims from traditional herbal medicines, such as tea.

The most well documented therapeutic effect of green tea concerns the antioxidant properties of a group of its components called polyphenols and in particular the subgroup called catechins. The powerful antioxidant properties of the catechins is what is responsible for most of the health benefits, including the prevention of oxidation of LDL cholesterol, vasorelaxation, anti-cancer properties and more.

Furthermore, green tea has other health benefits, not necessarily related to its antioxidant properties. These include the improvement of bone density, improved kidney health, improved liver health and weight loss.

Even before the appearance of recent scientific literature on this subject, many people have been consuming green tea products as either therapeutic herbal supplements or a refreshing drink or both. Green tea is one of the oldest drinks from countries such as Japan and China, where its health benefits have been revered for centuries.

The green tea used in conventional products, such as tea bags and ready made beverages are of varying qualities. Most contain caffeine and a polyphenol content of 7-10 mg per serving. Other components of green tea, which are not so desirable, can include fluoride and vitamin K1. Higher polyphenol content dictates a greater bitterness to the product. Drinks that contain sufficient polyphenols content necessary to achieve beneficial effects can be objectionable because of the intense bitterness of the catechins.

The natural flavorings available on the market today can also impart bitterness to the products. Tea beverages high in polyphenols and imbued with natural flavorings can be extremely unpalatable. Many products on the market today contain substantial quantities of sugar to mask the bitterness. Sugar, however, has many detrimental effects on health and its use can negate any health benefits achievable from the green tea polyphenols.

Thus, it is an object of this invention to reveal a green tea based composition such that it provides substantial beneficial and therapeutic effects when consumed yet has a pleasant flavor. It is a further object of this invention to enhance the therapeutic benefits of green tea by combining it with flavor enhancing additives, which themselves have beneficial therapeutic properties but which do not have the problems associated with the use of sugar.

### SUMMARY OF THE INVENTION

The inventors herein propose combining green tea extract with natural flavoring agents and one or more additives selected from the group consisting of extracts derived from the fruits of Lo Han, extracts derived from the leaves of Stevia and extracts derived from the leaves of Chinese Blackberry to produce an aqueous drink. The inventors have discovered that dietary supplements and functional beverages prepared in accordance with the foregoing produce a great tasting herbal product which possesses many beneficial therapeutic properties, including the antioxidant properties, anti-cancer, kidney, liver and heart health properties.

Alternatively, the foregoing herbal combination can be formulated into a toothpaste or dental gel by combining the foregoing active ingredients with a thickener or gel or they can be formulated into an effective mouthwash. These oral hygiene products produce a delicious and effective natural alternative to typical synthetic toothpastes and mouthwashes currently available.

### DETAILED DESCRIPTION OF THE INVENTION

The inventors herein propose a dietary supplement or nutritional beverage and/or oral hygiene product comprising:

(a) green tea extract;

(b) optionally, natural flavoring agent;

(c) at least one additive selected from the group consisting of (i) extracts of the fruits of Lo Han, (ii) extracts from the leaves of Stevia, and (iii) extracts from the leaves of Chinese Blackberry.

Additional optional ingredients include glycerin xylitol, fructose or agave syrup or other preferably low glycemic sugars, vitamins, minerals, other herbal extracts, stabilizers such as Chinese Licorice root extracts, and thickeners or gelling agents. The combination of the foregoing ingredients provides a great tasting dietary supplement, nutritional beverage or dental hygiene product, which possesses the therapeutic properties provided for herein.

The dietary supplement, nutritional beverage or dental hygiene product described herein requires a source of green tea. The inventors have found green tea extract to be a useful source of polyphenols for use in this invention. Green tea extract is preferred because of its concentrated form. In choosing a source of green tea, it is preferred that the polyphenol content range from about 50% to 98% and the caffeine content range from about 0.1% to 1%. Furthermore, fluoride and vitamin K1 should preferably be removed during the extraction process. The concentration of green tea in the dietary supplement or nutritional beverage, on a dry basis, may range from about 1% to 15% by weight. In the oral hygiene product, the concentration of green tea, on a dry basis, is preferably from about 1% to 5% by weight. Green tea generally comes from the leaves of a tea bush such as the Camellia Sinesis plant. Green tea leaves are steamed, or baked, shortly after plucking, however they are not oxidized or “fermented” as is done with black tea. Because green tea leaves are not fermented, the finished leaves are very similar to the natural leaves from the tea bush. Most of the world's green tea leaves are produced in either China or Japan. It is these green tea leaves that serve as the raw plant material for the green tea extract used in this invention.

The dietary supplement, nutritional beverage or oral hygiene product described herein also preferably comprises a natural flavoring. Natural flavors are derived from a spice, fruit, vegetable, herb or other similar natural material. Natural flavors generally comprise essential oil, essence or extractive, or distillate of the natural plant material it is derived from. Natural flavors include the natural essence or extractives obtained from plants listed in 21 CFR 182.10, 182.20, 182.40, 182.50 and 184, and the substances listed in 21 CFR 172.510, the disclosures each of which are incorporated herein by reference. The primary purpose of the natural flavor component is to provide or add flavor of a desired type. Typical extraction techniques can be used to derive the natural flavor components from the applicable natural plant material. It is preferable that the flavoring does not contain synthetic or artificial carriers, such as propylene glycol (liquid) or maltodextrin (powder). The concentration of the foregoing additives in the composition may range in concentration depending upon the type of flavors themselves. The concentration of the flavor components in the dietary supplement, nutritional beverage or oral hygiene product, on a dry basis, may range from about 1% to 10% by weight, or the equivalent to about 10 mg to 100 mg per serving. These kinds of flavors are readily available from raw material manufacturers. The flavors are usually custom made for the client but need not be so. One such company is the Virginia Dare Company located in Brooklyn, N.Y. Peach, orange, lemon and mango natural flavors are preferred.

The dietary supplement, nutritional beverage or oral hygiene product described herein requires at least one additive selected from the group consisting of (i) extracts from the fruits of Lo Han, (ii) extracts from the leaves of Stevia and (iii) extracts from the leaves of Chinese Blackberry. Preferably more than one or all of the foregoing additives are present in the composition. The concentration of the foregoing additives in the composition may range in concentration depending upon the concentration of the extracts themselves. Generally, these extracts are provided as dried powders and if so, the concentration of these additives in the composition of this invention will range from about 0.05% to 6% by weight but is preferably from about 0.05% to 1% by weight.

Lo Han fruit comes from Momordica grosvenorii also called Siraitia grosvenorii. Lo Han is native to the People's Republic of China and Japan and is an edible fruit having an intensely sweet taste. It is reputed to possess healing properties for lung congestion, colds, sore throats, digestive and urinary disturbances as well as antibiotic and antiseptic properties. Extracts of both fresh and dried Lo Han are available throughout the world and can be readily purchased. One suitable extract is available from the Fortune Bridge Company, located in New York. Various methods are known for preparing extracts of the Lo Han fruit such as those methods described in the U.S. Pat. Nos. 6,124,442 and 4,084,010, the teachings each of which are incorporated herein by reference in their entirety.

Stevia (Stevia rebaudiana also called Eupatorium rebaudiana) is also a plant, which is native to the People's Republic of China. Extracts from the leaves of Stevia are commonly known and have been used as a natural sweetener in Asia for many years. Studies have demonstrated that extracts from the leaves of Stevia can have a variety of beneficial therapeutic effects, including antibiotic properties (especially against E. coli), vasodilation properties (especially in the kidney), anti-hypertensive effects, beneficial effects on pancreatic beta cells and enhancing the secretion of insulin. Extracts from the leaves of Stevia are known and can be purchased for use in formulating the compositions described herein. These extracts can be prepared using the same or similar techniques used for preparing extracts for Lo Han. One suitable extract is available from the Fortune Bridge Company, located in New York.

Extracts from the leaves of Chinese Blackberry (Rubus suavissimus) are also known and can be purchased for use in formulating the compositions described herein. One suitable extract is available from the Fortune Bridge Company, located in New York. The active ingredients in these extracts are believed to comprise diterpene glycosides. Extracts of the leaves of Chinese Blackberry have been used as natural sweeteners. A variety of therapeutic benefits have been ascribed to extracts of the leaves of Chinese Blackberry, including relief from premenstrual syndrome, beneficial effects upon the kidney and other beneficial effects. Extracts of the leaves of Chinese Blackberry can be prepared using the same or similar techniques used for preparing extracts of Lo Han.

Various methods are known for preparing extracts of the Lou Han fruit, and other natural sources used in this invention, such as those methods described in U.S. Pat. Nos. 6,124,442 and 4,084,010, the teachings each of which are incorporated herein by reference in their entirety. Preferred extracts are prepared using water or water/alcohol mixtures to extract the active species from the plant, thereby creating the extract useful in this invention. One such preferred method is described in U.S. Pat. No. 6,124,442, wherein the starting plant source material is fresh plant material such as, in the case of Lou Han, cut pieces of freshly harvested or dried Lou Han fruit. In the case of Stevia, tea, and Chinese Blackberry, this would be primarily leaves, in the case of Chinese Licorice, this would be primarily root pieces. The applicable plant pieces are extracted by soaking in a bath of heated (80° F.-212° F.) water, alcohol (preferably ethanol) or both. This initial process is preferably carried out in hot, preferably boiling water or water/alcohol mixture. Extraction in the bath may be carried out several times, each time saving the resulting liquid. The resulting extract liquid is then filtered through a suitable filter, such as a 40-mesh stainless steel screen. The filtered out solid material can then be added back to the process for further extraction if desired. The result of the extraction process is an aqueous, alcohol or aqueous/alcohol extract that can be used in the composition of this invention. The extract can be used directly, or first concentrated by evaporation or spray drying and then used. Each of the extracts used in this invention can be prepared in this manner by starting with the appropriate material: Lou Han (fruit), Stevia (leaves), Tea (leaves), Chinese Blackberry (leaves) and Chinese Licorice (roots). If alcohol is used, alone or with water, it is preferably ethanol.

As noted, other optional materials may be included in the composition of this invention. Vitamins, minerals or other herbal extracts may be added for particular purposes or effects. Sugars, such as fructose may be added, but are not recommended. Polyol sweeteners, such as xylitol may be added. Preferably, the composition is substantially free of sucrose. Other flavoring agents, such as natural peach, mango, vanilla or other flavor may also be utilized. Glycerin has also proven to be a beneficial additive.

Stabilizers for the compositions of this invention may also be employed to preserve the flavor and other properties of the product. The inventors have found that extracts from the root of the Chinese Licorice (Glycyrrhiza uralensis) have proven to be excellent stabilizers for the products described herein as well as flavor enhancers. Extracts of the root of the Chinese Licorice are known and can be purchased for use in this invention. One suitable extract is available from the Fortune Bridge Company, located in New York. These extracts have been used for their beneficial medicinal properties for years. If used, the concentration of Chinese Licorice extract in the composition of this invention may range from 0.001% to 1.0% by weight. The useful Chinese Licorice extracts are prepared in the same manner as the Lou Han extracts and are preferably aqueous extracts of the Chinese Licorice root.

In preparing the compositions of this invention for use as dietary supplements or nutritional beverage, the ingredients noted herein are merely combined with purified water in the concentrations noted to form a dietary supplement drink. It is recommended that the consumer of the product ingest at least two or three servings of the diluted product per day, such that one receives 200 mg of polyphenols. In preparing the compositions described herein as toothpaste or a dental gel, the ingredients taught herein are combined with small quantities of water and appropriate thickeners or gelling agents.

The compositions of this invention are further described in the following examples, which should be taken as illustrative only and not limiting in any manner.

### EXAMPLE I

A green tea based dietary supplement (nutritional beverage) was prepared using the following ingredients:

| Component | % by Weight |
|---|---|
| Green tea extract¹ | 9 |
| Lo Han extract² | 3 |
| Stevia extract³ | 1.5 |
| Chinese Licorice extract⁴ | 0.2 |
| Natural peach flavor⁵ | 6 |
| Glycerin | 30 |
| Water | 50.3 |

¹Available from Fortune Bridge Company, NY
²Available from Fortune Bridge Company, NY
³Available from Fortune Bridge Company, NY
⁴Available from Fortune Bridge Company, NY
⁵Available from Virginia Dare Company, NY

The foregoing ingredients were blended to form a pleasant tasting dietary supplement (nutritional beverage) which is believed to exhibit the beneficial therapeutic effects noted herein.

### EXAMPLE II

Example I was repeated except that the concentrations of Stevia extract and Lo Han extract were reversed. The same results were achieved.

### EXAMPLE III

Example I was repeated except that Chinese Blackberry extract powder was substituted for the Stevia extract powder at the same concentrations. The same results were achieved.

### EXAMPLE IV

A green tea based dietary supplement (nutritional beverage) was prepared using the following ingredients:

| Component | % by Weight |
|---|---|
| Green tea extract⁶ | 9 |
| Lo Han extract⁷ | 3 |
| Fructose | 1.5 |
| Chinese Licorice extract⁸ | 0.2 |
| Natural peach flavor⁹ | 6 |
| Glycerin | 30 |
| Water | 50.3 |

⁶Available from Fortune Bridge Company, NY
⁷Available from Fortune Bridge Company, NY
⁸Available from Fortune Bridge Company, NY
⁹Available from Virginia Dare Company, NY

The foregoing ingredients were blended to form a pleasant tasting dietary supplement which is believed to exhibit the beneficial therapeutic effects noted herein.

### EXAMPLE V

A green tea based dental gel was prepared using the following ingredients:

| Component | % by Weight |
|---|---|
| Green tea extract¹⁰ | 5 |
| Lo Han extract¹¹ | 3 |
| Chinese Licorice extract¹² | 0.2 |
| Natural peach flavour¹³ | 6 |
| Gel base | 85.8 |

¹⁰Available from Fortune Bridge Company, NY
¹¹Available from Fortune Bridge Company, NY
¹²Available from Fortune Bridge Company, NY
¹³Available from Virginia Dare Company, NY

The foregoing ingredients were blended to form a beneficial dental gel.

1. A composition useful as a dietary supplement or an oral hygiene product, which composition comprises:
(a) a source of green tea extract;
(b) at least one additive selected from the group consisting of (i) extracts of the fruits of Lo Han, (ii) extracts of the leaves of Stevia and (iii) extracts of the leaves of Chinese Blackberry; and
(c) optionally, a natural flavoring component.

2. A composition according to claim 1 wherein the composition also comprises at least one material selected from the group consisting of extract of Chinese Licorice root, vitamins, fructose, glycerin, thickeners, gelling agents, xylitol.

3. A composition according to claim 1 wherein the composition is substantially free of sucrose.

4. A composition according to claim 1 wherein the concentration of the source of green tea, on a dry basis, is from about 1% to 15% by weight.

5. A composition according to claim 1 wherein the concentration of the additive(s), on a dry basis, is from about 0.05% to 5.0% by weight.

6. A composition according to claim 2 wherein the concentration of extract of Chinese Licorice root is from about 0.001% to 1.0% by weight.

7. A composition according to claim 2 wherein the composition is substantially free of sucrose.

8. A composition according to claim 2 wherein the concentration of the source of green tea, on a dry basis, is from about 1% to 15% by weight.

9. A composition according to claim 2 wherein the concentration of the additive(s) on a dry basis, is from about 0.05% to 5.0% by weight.
